(19) United States
(12) Patent Application Publication
(10) Pub. No.: US 2023/0349119 A1
(43) Pub. Date: Nov. 2, 2023

(54) SYSTEMS, METHODS, AND COMPUTER PROGRAM PRODUCTS FOR DELIVERING FLUID AT A WORKSITE

(71) Applicant: Caterpillar Paving Products Inc., Brooklyn Park, MN (US)

(72) Inventors: Nicholas A. OETKEN, Brooklyn Park, MN (US); Mark D. BEHN, Saint Michael, MN (US); Nathaniel S. DOY, Maple Grove, MN (US); Robert K. IVERSON, Maple Grove, MN (US); John Lee MARSOLEK, Watertown, MN (US); Brian D. NAGEL, Ramsey, MN (US); Kishore Sundara SRINIVASAN, Chennai (IN)

(73) Assignee: Caterpillar Paving Products Inc., Brooklyn Park, MN (US)

(21) Appl. No.: 17/730,186

(22) Filed: Apr. 27, 2022

(51) Int. Cl. E02D 3/10; G01N 33/24; G05D 1/02; E02D 1/02

(52) U.S. Cl. CPC E02D 3/10; G01N 33/246; G05D 1/0219; G05D 1/0276; E02D 1/027; E02D 2600/10; E02D 3/026

(57) ABSTRACT

Systems, methods, and computer program products can wirelessly receive moisture data outputted from one or more wireless moisture sensors at least partially embedded in a material to be compacted by a compaction machine; determine, based on the moisture data from the one or more wireless moisture sensors, whether water needs to be added to the material at each of the one or more wireless moisture sensors; and output signaling to direct a water truck to provide water to the portion or portions of the material determined to need water.

### TECHNICAL FIELD

The present disclosure relates to systems, methods, and computer program products for delivering fluid at a worksite, particularly delivering fluid at the worksite for at least the purpose of compacting a material at the worksite.

### BACKGROUND

Generally, ground material, such as soil, may need to have a certain amount of moisture in order to provide lubrication during a compaction process performed by a compactor machine. This can allow the material particles to move into a compacted state more easily. In this regard, it may be desirable to keep the moisture content of the material to within a certain range around an optimum moisture content, which may be identified in advance during a proctor testing, to maximize the dry density of the material after compacting. However, the moisture content of the material may vary across a jobsite. This can mean that the compactor machine operator may not be aware whether the moisture content of the material is within the proper range for suitably performing the compacting.

U.S. Pat. No. 11,168,454 (“the '454 patent”) describes a method for moisturizing soil at an open construction site. The '454 patent describes that the method includes determining a target soil moisture level for the soil at the open construction site, measuring a current soil moisture level of a location within the open construction site with a moisture sensor while the moisture control system is moving along a predetermined path across the site, storing the current soil moisture level of the location in memory, determining a target volume of water for achieving the target soil moisture level at the location based on the current soil moisture level at the location, calculating a target application rate to achieve the target volume of water, and applying the target volume of water at the target application rate to the location when the system is positioned to dispense water at the location of the site. The '454 patent further describes that a dynamic soil moisture control system is implemented in a water truck.

### SUMMARY

According to an aspect of the present disclosure, a method is described or implemented. The method can comprise: wirelessly receiving, at processing circuitry, moisture data outputted from one or more wireless moisture sensors at least partially embedded in a material to be compacted by a compaction machine at a jobsite; identifying, using the processing circuitry, whether the material around each of the one or more wireless moisture sensors qualifies for compaction according to predetermined criteria, the predetermined criteria for compaction by the compaction machine including whether a moisture content of the material around each of the one or more wireless moisture sensors is within a predetermined moisture threshold range; and responsive to the material around at least one of the one or more wireless moisture sensors failing to qualify for compaction, wirelessly outputting, using the processing circuitry, signaling to direct a water truck to provide water to a surface of the material associated with a location of the at least one wireless moisture sensor.

According to another aspect of the present disclosure, a non-transitory computer-readable storage medium is described or can be implemented. The non-transitory computer-readable storage medium can have stored thereon instructions that, when executed by one or more processors, cause the one or more processors to perform a method. The method can comprise: wirelessly receiving moisture data outputted from at least one wireless moisture sensor at least partially buried in a material to be compacted by a compactor at a worksite; determining, based on the moisture data, whether the material at each said at least one wireless moisture sensor is or will soon be below a predetermined moisture threshold range; and responsive to said determining indicating that the material at each said at least one wireless moisture sensor is or will soon be below the predetermined moisture threshold range, outputting signaling to direct a water truck to provide water to a surface of the material associated with each said at least one wireless moisture sensor.

According to another aspect of the present disclosure, a system for compacting soil based on moisture content of the soil is described or can be implemented. The system can comprise: a plurality of wireless moisture sensors at least partially embedded in the soil at spaced relationships throughout at least a portion of a worksite to sense the moisture content of the soil around the wireless moisture sensors; a water truck to provide water to the soil based on moisture data periodically outputted from the wireless moisture sensors; and a control station different from the water truck. The control station including circuitry can be configured to wirelessly receive the moisture data outputted from the wireless moisture sensors, identify, based on the moisture data outputted from the wireless moisture sensors, whether the moisture content of the soil around each of the wireless moisture sensors is below a predetermined moisture threshold, and for each of the wireless moisture sensors, in a case where the moisture content of the soil around the wireless moisture sensor is identified to be below the predetermined moisture threshold, output signaling to direct the water truck to provide water to the soil around the wireless moisture sensor to increase the moisture content of the soil to at least the predetermined moisture threshold.

### BRIEF DESCRIPTION OF DRAWINGS

FIG. 1 is a diagram of a portion of a system according to one or more embodiments of the disclosed subject matter.

FIG. 2 and FIG. 3 show views of another portion of the system of FIG. 1 according to one or more embodiments of the disclosed subject matter.

FIG. 4 and FIG. 5 show views of a variation of the portion of the system of FIG. 2 and FIG. 3, respectively.

FIG. 6 is a flow chart of a method according to one or more embodiments of the disclosed subject matter.

### DETAILED DESCRIPTION

The present disclosure relates to systems, methods, and computer program products for delivering fluid at a worksite, particularly delivering fluid at the worksite for at least the purpose of compacting a material at the worksite. Thus, the present disclosure can also relate to systems, methods, and computer program products for compacting material at a worksite.

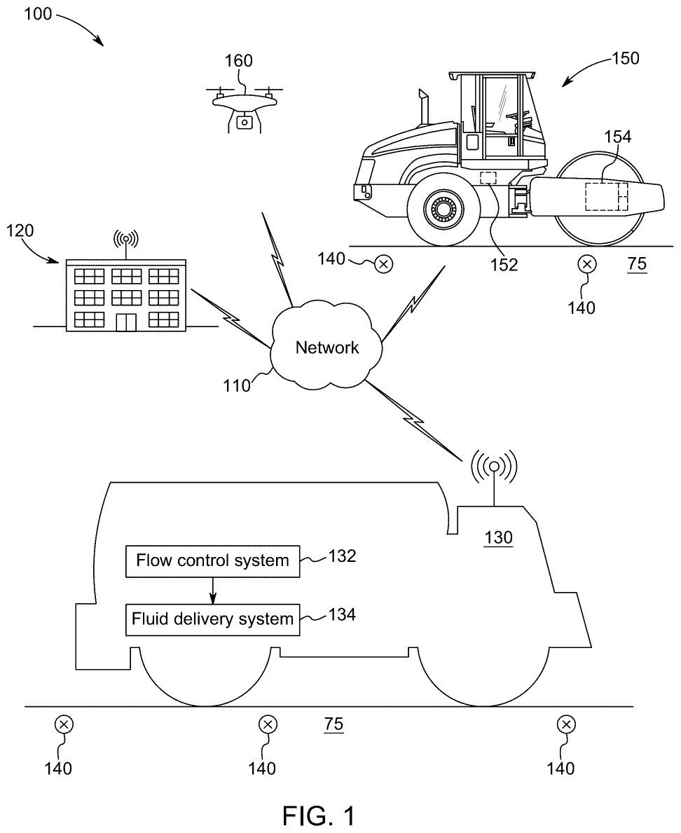

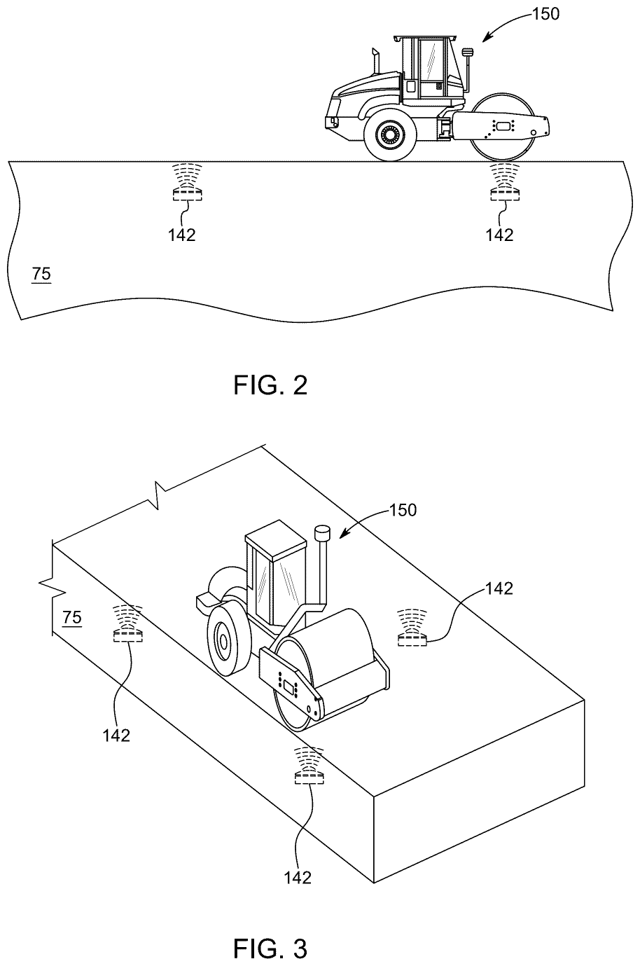

FIG. 1 is a diagram of a portion of a system 100 according to one or more embodiments of the disclosed subject matter. FIG. 2 and FIG. 3 show views of another portion of the system 100 of FIG. 1.

Generally, the system 100 can pertain to a worksite or jobsite 50 and can include a base or control station 120, one or more water trucks 130, and one or more moisture sensors 140. The system 100 may also be comprised of one or more compactor or compaction machines 150. The water truck 130 and/or the compactor 150 can be autonomous or semi-autonomous, according to one or more embodiments of the disclosed subject matter.

The system 100 may be referred to or characterized as a system to provide or deliver a fluid (e.g., water) to the worksite 50, particularly to material 75 of the worksite 50, at least for the purpose of compacting the material 75. Additionally or alternatively, the system 100 may be referred to or characterized as a system to compact the material 75, for instance, using the compactor 150. The material may be ground or earthen material, such as soil, dirt, sand, clay, etc. According to one or more embodiments, the material 75 may not be asphalt or some other material that does not need application of water for lubrication of material particles for compaction.

The control station 120 can wirelessly communicate with the water truck 130 and the compactor 150 via a wireless network 110, where each of the control station 120, the water truck 130, and the compactor 150 can include circuitry (e.g., control circuitry (including processing circuitry) 152, communication circuitry, sensing circuitry 154, etc.). As examples, the wireless network 110 can be or include a cellular network and/or a local area network (e.g., Wi-Fi).

The control station 120 can be local to the worksite 50 and/or remote from the worksite 50. According to one or more embodiments, in the case of the control station 120 being remote from the worksite 50, the control station 120 may be referred to or characterized as a back office system. The control station 120 may be cloud-based, according to one or more embodiments of the disclosed subject matter. Thus, data from the worksite 50 may be referred to or characterized as being sent to the cloud when sent to the cloud-based control station 120.

The moisture sensors 140 can be in wireless communication with one or more electronic devices at the worksite 50. Such wireless communication can be via a wireless network or system different from the wireless network 110 and/or via the wireless network 110. For instance, the communication different from the wireless network 110 may be via radio-frequency identification (RFID). The one or more electronic devices at the worksite 50 can be or include the compactor 150 (i.e., circuitry thereof); the control station 120, when local to the worksite 50, or local components thereof, such as a node (e.g., wireless router, etc.); and/or a sensing machine 160 such as a drone or unmanned aerial vehicle (UAV). According to one or more embodiments, ultimately, communications from the moisture sensors 140 can be received at the control station 120, either directly (including the local component(s) of the control station 120) or indirectly, for instance, via the compactor 150 and/or the sensing machine 160. According to one or more embodiments, the compactor 150 communicating directly with the moisture sensors 140 can be referred to or characterized as a sensing machine.

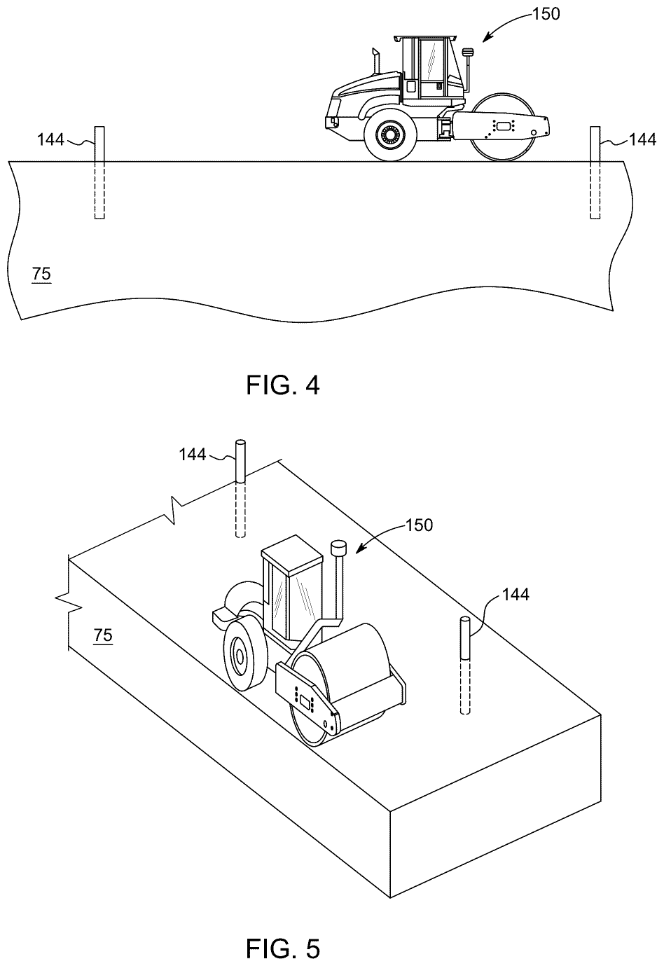

With reference to FIG. 2 and FIG. 3 and also FIG. 4 and FIG. 5, the moisture sensors 140 can sense moisture content of the material 75. In particular, the moisture sensors 140 can sense moisture content at least at a predetermined distance below a surface of the material 75, that is, at a subsurface of the material 75. The surface of the material 75 can be a surface over which the compactor 150 move to compact the material 75. Further, the moisture sensors 140 may be provided, i.e., at least partially embedded in the material 75 at the worksite 50, at spaced positions about the worksite 50 relative to each other. Such spaced positioning may be uniform or irregular. According to one or more embodiments, each moisture sensor 140 may be at least a predetermined distance away from all other moisture sensors 140 provided at the worksite 50 in a direction of the surface of the material 75.

At least when the moisture sensor 140 is initially placed, a sensing portion of the moisture sensor 140 can be at or below a predetermined depth from the surface of the material 75. According to one or more embodiments, the sensing portions of different moisture sensors 140 can be placed at the same depth or at different depths. For instance, sensing portions of the moisture sensors 140 can be placed 6 to 12 inches below the surface of the material 75. According to one or more embodiments, the depth at which the sensing portion of the moisture sensor 140 is placed can be based on the transmission capability (e.g., power) of the circuitry of the moisture sensor 140.

Here, the moisture sensors 140 can directly sense the moisture content of the material 75, for instance, by measuring resistance between two predetermined portions of the moisture sensor 140 and outputting a corresponding electrical signal representative of the moisture content. As examples, the sensing portion of the moisture sensor 140 may be or include a dual- or single-type probe. Such direct sensing can be rather than sensing being performed based on a ground penetrating radar output by a machine at the surface of the material 75, for instance. In this regard, according to one or more embodiments, sensing portions of the moisture sensors 140 may be placed deeper than a depth to which ground penetrating radar may reach.

The depths of the moisture sensors 140 may be known upon initial placement in the material 75 and saved, for instance, at the control station 120. It is noted that in certain cases, depending upon the type of moisture sensor 140, the depth at which the moisture sensor 140 is provided may change as the compactor 150 compacts the material. For instance, the moisture sensor 140 may be initially provided in the material 75 at a depth of 12 inches below the surface of the material 75 and end up, due to compaction of the material 75 by the compactor 150 at a depth of 8 to 10 inches below the surface of the material 75. The moisture sensor 140 can remain operative even if its depth changes. According to one or more embodiments, the moisture sensors 140 may remain in the ground after compacting by the compactor 150.

FIG. 2 and FIG. 3 show moisture sensors 140 in the form of tags, i.e., tag moisture sensors 142. In the case of tag moisture sensors 142, such moisture sensors can be fully embedded or buried in the material 75. For instance, the tag moisture sensors 142 can be placed 6 to 12 inches below the surface of the material 75. As noted above, not all of the tag moisture sensors 142 can be at the same depth, though, according to embodiments of the disclosed subject matter, all of the tag moisture sensors 142 can be within the same depth range (e.g., 6 to 12 inches). Here, the tag moisture sensors 142 can directly sense moisture of the material 75 around or at the moisture sensor 142. As an example, the tag moisture sensors 142 may include radio-frequency identification (RFID) circuitry and thus communicate via RFID, though embodiments of the disclosed subject matter are not limited to RFID communication for the tag moisture sensors 142.

FIG. 4 and FIG. 5 show moisture sensors 140 in the form of probes or stakes, i.e., stake moisture sensors 144. In the case of stake moisture sensors 144, such moisture sensors can be partially embedded or buried in the material 75 such that a portion of the stake moisture sensor 144 extends from the surface of the material 75. A sensing portion of the stake moisture sensor 144 can be below the surface of the material 75. For instance, the sensing portion of the stake moisture sensors 144 can be placed 6 to 12 inches below the surface of the material 75. Not all of the sensing portions of the stake moisture sensors 142 can be at the same depth, though, according to embodiments of the disclosed subject matter, though all of the sensing portions of the stake moisture sensors 144 can be within the same depth range (e.g., 6 to 12 inches). The sensing portions of the stake moisture sensors 144 can directly sense moisture of the material 75 around or at the moisture sensor 142. As an example, the stake moisture sensors 144 may include local area network (e.g., Wi-Fi) circuitry and thus communicate via a local area network, though embodiments of the disclosed subject matter are not limited to local area network communication for the stake moisture sensor 144.

The moisture sensors 140 can sense or detect moisture content of the surrounding material 75 and output moisture data. Such outputting can be continuous or intermittent. For instance, according to one or more embodiments, the moisture sensors 140 may output moisture data every five minutes. According to one or more embodiments, the moisture data output by the moisture sensor 140 can be received, for instance, by a machine (e.g., compactor 150 and/or sensing machine 160) only when the machine is within a predetermined distance from the moisture sensor 140. Further, the moisture sensor 140 may transmit the moisture data only when the machine is within the predetermined distance from the moisture sensor 140. This may involve two-way communication between the moisture sensor 140 and the machine (e.g., handshake, RFID trigger, etc.). In the case of the machine being within the predetermined distance from the moisture sensor 140, the outputting of the moisture data can occur every 10 seconds to every minute, as an example. Such predetermined distance may be based on the communication system between the moisture sensor 140 and the machine (e.g., based on transmission range, such as RFID technology). Optionally, the moisture sensor 140 may output moisture data only when moisture content of the material 75 is detected to be below a predetermined moisture threshold (e.g., moisture or water content of 6%).

As noted above, the moisture data from the moisture sensor 140, or processed versions thereof, can be output from the machine (e.g., compactor 150 and/or sensing machine 160) to the control station 120. Based on transmit and receive time to obtain the moisture data, the machine may estimate a depth of the moisture tag 140 and send this information also to the control station 120. The control station 120 can use this information to correlate moisture content to a specific depth of the material 75.

Since the stake moisture sensors 144 can stick out of material 75 (e.g., communication circuitry thereof can be in the above-surface portion), the stake moisture sensors 144 may have a greater transmit distance, for instance, compared to the tag moisture sensors 142, which can be fully embedded in the material 75. For instance, the stake moisture sensors 144 may be able to communicate (including sending moisture data) directly to the control station 120, whereas the tag moisture sensors 142 may need to communicate with the control station 120 via an intermediate electronic device, for instance, circuitry of the compactor 150 and/or circuitry of the sensing machine 160, which can relay (and optionally process) the moisture data from the tag moisture sensor 142 to the control station 120.

The control station 120 can wirelessly receive moisture data from the moisture sensors 140, either directly or indirectly, as discussed above. Based on the received moisture data, the control station 120, i.e., circuitry thereof, can determine or identify whether fluid (e.g., water or a water solution) needs to be added to the material 75 around or at the moisture sensors 140, via application of the fluid to the surface of the material 75. According to an alternative embodiment, the machine (e.g., the compactor machine 150 and/or the sensing machine 160) can determine or identify whether fluid needs to be added to the material 75 around or at the moisture sensors 140 and transmit this information to the control station 120. Thus, according to one or more embodiments, the machine may be considered part of the control station 120. In any event, some or all of the moisture sensors 140 may output moisture data indicative of the need to provide fluid to associated portions of the material 75.

Determining whether fluid needs to be added to the material 75 around or at the moisture sensors 140, via application of the fluid to the surface of the material 75, can include determining whether the moisture content of the material 75 around the moisture sensor 140 is below or will soon be (e.g., within a predetermined estimated time period) below a predetermined moisture threshold (e.g., moisture or water content of 6%). Here, the predetermined moisture threshold may be a lower boundary of a predetermined moisture threshold range (e.g., water content of at or about 6% to at or about 10%).

The predetermined moisture threshold (and predetermined moisture threshold range) can be set based on preferred (including optimum) dry unit weight (density) of the material 75 for the purpose of compacting the material 75 using the compactor 150. Here, too little fluid may make it more difficult to move the material particles during compaction and too much fluid may displace the material particles. The two extremes may set the boundaries of the predetermined moisture threshold range, with the range including an optimum moisture content (e.g., at or about 8%) to maximize dry unit weight (density) of the material 75. Thus, whether the moisture content of the material 75 associated with a particular moisture sensor 140 is below the predetermined moisture threshold may indicate or identify that the material 75 around the moisture sensor 140 does not qualify for compaction. Incidentally, likewise, the moisture content of the material 75 being above the predetermined moisture threshold range may indicate or identify that the material 75 around the moisture sensor 140 also does not qualify for compaction. Put another way, if the moisture content of the material 75 associated with the moisture sensor 140 is above the predetermined moisture threshold (including within the predetermined moisture threshold range), this may indicate, or the control station 120 may identify, that the associated portion of the material 75 qualifies for compaction by the compactor 75, for instance, due to the dry unit weight (density) being acceptable (including maximized) for compaction. Thus, according to embodiments of the disclosed subject matter, the compactor 150 may be selectively controlled to compact the material 75 when the moisture content of the material 75 has been determined to reach the upper limit of the predetermined moisture threshold range.

Based on identification of whether fluid (e.g., water or a water solution) needs to be added to the material 75 around or at the moisture sensors 140, via application of the fluid to the surface of the material 75, the water truck 130 can be controlled (e.g., by a driver or autonomously) to provide the fluid to the surface of the material 75 at the particular portion or portions associated with the moisture sensor(s) 140 indicating the need for the fluid. According to one or more embodiments, the control station 120 can wirelessly output signaling to direct the water truck 130 to the location or locations where the fluid is needed. Such signaling may be automatically performed responsive to identification of fluid needing to be added to the material 75 around or at the moisture sensors 140.

The output signaling may also indicate how much fluid to provide at each particular location. The amount of fluid to provide may be to place the moisture content of the material 75 around the particular moisture sensor 140 to at least above the predetermined moisture threshold, to within the predetermined moisture threshold range, or even to above the predetermined moisture threshold range in the case that it is estimated that the moisture content will be within the predetermined moisture threshold range by the time the compactor 150 arrives to compact this portion of the material 75. Applying fluid to the surface of the material 75 so the fluid seeps into the sublayer of the material 75 may be referred to or characterized as making a moisture adjustment to the material 75.

The signaling to direct the water truck 130 can be to provide information (e.g., location, amount, timing, etc.) on a display of the water truck 130 for an operator of the water truck 130 to refer to during operation of the water truck 130. Alternatively, the signaling can be or include commands to actually control the water truck 130, for instance, in a case where the water truck 130 is operating autonomously, to the apply the fluid at the desired location, in the desired amount, at a desired timing, etc. Thus, the water truck 130 can be directed to provide fluid to the material 75 to place or maintain the material 75 in suitable form for compaction by the compactor 150. The water truck 130 may include a flow control system 132 and a fluid delivery system 134 to output the fluid from the water truck 130. The flow control system 132 may receive control signaling based on the signaling to direct the water truck 130, for instance, from the operator of the water truck 130 or under autonomous control, which can output further control signaling to control the fluid delivery system 134 and the actual output of the fluid.

According to one or more embodiments, the control station 120 may estimate how much fluid is in the material 75 around each moisture sensor 140, by gathering weather information (e.g., sunny, rainy, humidity, cloudy, precipitation, etc.), current and/or future, to estimate the moisture content of the material 75. Such moisture content estimate may, in turn, be used to determine when, where, and/or how much regarding fluid to add to the material 75. The estimate may also be based on material type (e.g., soil type). The control station 120 may perform the estimate between receipt of moisture data from the moisture sensors 140, such as when the moisture data has not been received within a predetermined amount of time.

The compactor 150 may compact the material 75 when the material 75 is suitable for compaction. Suitable for compaction can mean that the moisture content of the material 75 is at least above the predetermined moisture threshold or within the predetermined moisture threshold range. As noted above, the predetermined moisture threshold (and predetermined moisture threshold range) can be set based on preferred (including optimum) dry unit weight (density) of the material 75 for the purpose of compacting the material 75 using the compactor 150.

According to one or more embodiments, the compactor 150 may wirelessly receive signaling from the control station 120 regarding which portion or portions of the material 75 to compact. Such portion or portions of the material 75 may have been identified by the control station 120 as being suitable for compaction. As noted above, this can mean that the moisture content of the material 75 is or will soon be (e.g., the water truck 130 has been directed to apply fluid prior to the arrival of the compactor 150) at least above the predetermined moisture threshold or within the predetermined moisture threshold range.

The signaling to direct the compactor 150 can be to provide information (e.g., location, amount, timing, etc.) on a display of the compactor 150 for an operator of the compactor 150 to refer to during operation of the compactor 150. Alternatively, the signaling can be or include commands to actually control the compactor 150, for instance, in a case where the compactor 150 is operating autonomously, to the compact the material 75 at a desired location, according to a desired timing, etc.

### INDUSTRIAL APPLICABILITY

As noted above, the disclosed subject matter relates to systems, methods, and computer program products for delivering fluid at a worksite, particularly delivering fluid at the worksite for at least the purpose of compacting a material at the worksite. The disclosure subject matter also relates to systems, methods, and computer program products for compacting material at a worksite.

Also noted above, it may be desirable to keep the moisture content of a material at a worksite or jobsite to within a certain range around an optimum moisture content, which may be identified in advance during a proctor testing, to maximize the dry density of the material after compacting. Thus, embodiments of the disclosed subject matter can, generally speaking, monitor the moisture content of the material using wireless moisture sensors at least partially buried about the worksite.

The moisture sensors can be wireless stakes or tags are installed into the ground surface of the worksite. The moisture sensors can measure the moisture content and send results to a nearby machine or transmit to a computer (local or cloud-based). The measured moisture content can be used to direct a water truck on the worksite to an area or areas of the worksite determined to require more fluid (e.g., water or water-based solution).

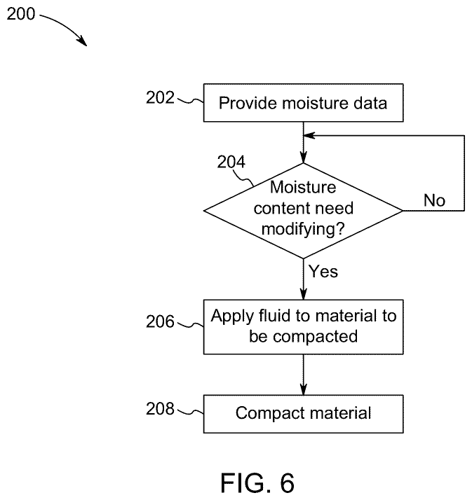

FIG. 6 is a basic flow chart of a method 200 according to one or more embodiments of the disclosed subject matter. The method 200 may be implemented via a non-transitory computer-readable storage medium having stored thereon instructions that, when executed by one or more processors or controllers, cause the one or more processors or controllers to perform the control method 200. Circuitry of the control station 120 may perform some or all of the operations of the method 200. Optionally, circuitry of the moisture sensor(s) 140, circuitry of the water truck 130, circuitry of the compactor 150, and/or circuitry of the sensing machine 160 can perform at least some of the method 200 or complements thereof (e.g., transmitting moisture data to the receiving control station 120, compactor 150, and/or sensing machine 160).

At 202 the method 200 can include providing moisture data regarding moisture content of the material 75. This can include wirelessly receiving moisture data outputted from one or more wireless moisture sensors 140 at least partially embedded in the material 75. The control station 120 can wirelessly receive moisture data from the moisture sensors 140, either directly or indirectly. For instance, the control station 120 can receive the moisture data from the moisture sensors 140 via the compactor 150 and/or the sensing machine 160, where the compactor 150 and/or the sensing machine 160 can receiving the moisture data from the moisture sensors 140 as the compactor 150 and/or sensing machine 160 move about the worksite 50.

At 204, the method 200 can determine or identify, based on the received moisture data, whether fluid (e.g., water or a water solution) should be added to the material 75 around or at the moisture sensors 140, via application of the fluid to the surface of the material 75. The control station 120 can make such determination. According to an alternative embodiment, a machine (e.g., the compactor machine 150 and/or the sensing machine 160) can determine or identify whether fluid needs to be added to the material 75 around or at the moisture sensors 140 and transmit this information to the control station 120. In any event, some or all of the moisture sensors 140 may output moisture data indicative of the need to provide fluid to associated portions of the material 75.

If at 204 it is determined that fluid should be added to the material 75, the method 200, at 206, can provide the water truck 130 (or some other fluid-providing vehicle) to provide the fluid to the portion or portions of the material 75 determined to be in need. Here, the water truck 130 can be controlled (e.g., by a driver or autonomously) to provide the fluid to the surface of the material 75 at the particular portion or portions associated with the moisture sensor(s) 140 that indicated the need for the fluid. According to one or more embodiments, the control station 120 can wirelessly output signaling to direct the water truck 130 to the location or locations where the fluid is needed. Such signaling may be automatically performed responsive to identification of fluid needing to be added to the material 75 around or at the moisture sensors 140.

The output signaling may also indicate how much fluid to provide at each particular location. The amount of fluid to provide may be to place the moisture content of the material 75 around the particular moisture sensor 140 to at least above the predetermined moisture threshold, to within the predetermined moisture threshold range, or even to above the predetermined moisture threshold range in the case that it is estimated that the moisture content will be within the predetermined moisture threshold range by the time the compactor 150 arrives to compact this portion of the material 75.

The signaling to direct the water truck 130 can be to provide information (e.g., location, amount, timing, etc.) on a display of the water truck 130 for an operator of the water truck 130 to refer to during operation of the water truck 130. Alternatively, the signaling can be or include commands to actually control the water truck 130, for instance, in a case where the water truck 130 is operating autonomously, to the apply the fluid at the desired location, in the desired amount, at a desired timing, etc. Thus, the water truck 130 can be directed to provide fluid to the material 75 to place or maintain the material 75 in suitable form for compaction by the compactor 150.

At 208, the method 200 can include compacting the material 75. Such compaction can be by way of the compactor 150 and can be for portions of the material 75 determined suitable for compaction. Suitable for compaction can mean that the moisture content of the material 75 is at least above the predetermined moisture threshold or within the predetermined moisture threshold range.

According to one or more embodiments, the compactor 150 may wirelessly receive signaling from the control station 120 regarding which portion or portions of the material 75 to compact. Such portion or portions of the material 75 may have been identified by the control station 120 as being suitable for compaction. As noted above, this can mean that the moisture content of the material 75 is or will soon be (e.g., the water truck 130 has been directed to apply fluid prior to the arrival of the compactor 150) at least above the predetermined moisture threshold or within the predetermined moisture threshold range.

The signaling to direct the compactor 150 can be to provide information (e.g., location, amount, timing, etc.) on a display of the compactor 150 for an operator of the compactor 150 to refer to during operation of the compactor 150. Alternatively, the signaling can be or include commands to actually control the compactor 150, for instance, in a case where the compactor 150 is operating autonomously, to the compact the material 75 at a desired location, according to a desired timing, etc.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present disclosure may take the form of a computer program product embodied in one or more computer readable medium(s) having computer readable program code embodied thereon.

The functionality of the elements disclosed herein may be implemented using circuitry or processing circuitry which includes general purpose processors, special purpose processors, integrated circuits, ASICs (“Application Specific Integrated Circuits”), conventional circuitry and/or combinations thereof which are configured or programmed to perform the disclosed functionality. Processors are considered processing circuitry or circuitry as they include transistors and other circuitry therein. The processor may be a programmed processor which executes a program stored in a memory. In the disclosure, the circuitry, units, or means are hardware that carry out or are programmed to perform the recited functionality. The hardware may be any hardware disclosed herein or otherwise known which is programmed or configured to carry out the recited functionality. When the hardware is a processor which may be considered a type of circuitry, the circuitry, means, or units are a combination of hardware and software, the software being used to configure the hardware and/or processor.

Further, as used herein, the term “circuitry” can refer to any or all of the following: (a) hardware-only circuit implementations (such as implementations in only analog and/or digital circuitry); (b) to combinations of circuits and software (and/or firmware), such as (as applicable): (i) a combination of processor(s) or (ii) portions of processor(s)/software (including digital signal processor(s)), software and memory(ies) that work together to cause an apparatus, such as a mobile phone or server, to perform various functions); and (c) to circuits, such as a microprocessor(s) or a portion of a microprocessor(s), that require software or firmware for operation, even if the software or firmware is not physically present. This definition of “circuitry” can apply to all uses of this term in this application, including in any claims. As a further example, as used in this application, the term “circuitry” can also cover an implementation of merely a processor (or multiple processors) or portion of a processor and its (or their) accompanying software and/or firmware.

Use of the terms “data,” “content,” “information” and similar terms may be used interchangeably, according to some example embodiments of the present disclosure, to refer to data capable of being transmitted, received, operated on, and/or stored. The term “network” may refer to a group of interconnected computers or other computing devices. Within a network, these computers or other computing devices may be interconnected directly or indirectly by various means including via one or more switches, routers, gateways, access points or the like.

Aspects of the present disclosure have been described above with reference to flowchart illustrations and/or block diagrams of methods, apparatus (systems) and computer program products according to embodiments of the present disclosure. In this regard, the flowchart and block diagrams in the Figures illustrate the architecture, functionality, and operation of possible implementations of systems, methods and computer program products according to various embodiments of the present disclosure. For instance, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It also will be understood that each block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks. The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

It must be noted that, as used in the specification and the appended claims, the singular forms “a,” “an,” and “the” include plural referents unless the context clearly dictates otherwise. That is, unless clearly specified otherwise, as used herein the words “a” and “an” and the like carry the meaning of “one or more.” The use of the term “at least one” followed by a list of one or more items (for example, “at least one of A and B” or one or more of A and B”) is to be construed to mean one item selected from the listed items (A or B) or any combination of two or more of the listed items (A and B; A, A and B; A, B and B), unless otherwise indicated herein or clearly contradicted by context. Similarly, as used herein, the word “or” refers to any possible permutation of a set of items. For example, the phrase “A, B, or C” refers to at least one of A, B, C, or any combination thereof, such as any of: A; B; C; A and B; A and C; B and C; A, B, and C; or multiple of any item such as A and A; B, B, and C; A, A, B, C, and C; etc.

Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein, merely describe points of reference and do not necessarily limit embodiments of the disclosed subject matter to any particular orientation or configuration. Furthermore, terms such as “first,” “second,” “third,” etc., merely identify one of a number of portions, components, points of reference, operations and/or functions as described herein, and likewise do not necessarily limit embodiments of the disclosed subject matter to any particular configuration or orientation.

While aspects of the present disclosure have been particularly shown and described with reference to the embodiments above, it will be understood by those skilled in the art that various additional embodiments may be contemplated by the modification of the disclosed machines, assemblies, systems, and methods without departing from the spirit and scope of what is disclosed. Such embodiments should be understood to fall within the scope of the present disclosure as determined based upon the claims and any equivalents thereof.

1. A system for compacting soil based on moisture content of the soil comprising:
a plurality of wireless moisture sensors at least partially embedded in the soil at spaced relationships throughout at least a portion of a worksite to sense the moisture content of the soil around the wireless moisture sensors;
a water truck to provide water to the soil based on moisture data periodically outputted from the wireless moisture sensors; and
a control station different from the water truck, the control station including circuitry configured to
wirelessly receive the moisture data outputted from the wireless moisture sensors,
identify, based on the moisture data outputted from the wireless moisture sensors, whether the moisture content of the soil around each of the wireless moisture sensors is below a predetermined moisture threshold, and
for each of the wireless moisture sensors, in a case where the moisture content of the soil around the wireless moisture sensor is identified to be below the predetermined moisture threshold, output signaling to direct the water truck to provide water to the soil around the wireless moisture sensor to increase the moisture content of the soil to at least the predetermined moisture threshold.

2. The system according to claim 1, further comprising a compaction machine,
wherein the circuitry of the control station is configured to wirelessly output information to the compaction machine regarding which portions of the soil are suitable for compaction based on the moisture content being determined to be within a predetermined moisture threshold range, the predetermined moisture threshold range having the predetermined moisture threshold as a lower range boundary, and
wherein the compaction machine is controlled to compact the portion or portions of the soil indicated as suitable for compaction based on the wirelessly output information output by the control station.

3. The system according to claim 1, wherein the wireless moisture sensors are partially embedded in the soil and are in the form of stakes.

4. The system according to claim 1, wherein the wireless moisture sensors are fully embedded in the soil and are in the form of tags.

5. The system according to claim 1,
wherein at least some of the wireless moisture sensors are partially embedded in the soil and are in the form of stakes, and
wherein at least some of the wireless moisture sensors are fully embedded in the soil and are in the form of tags.

6. The system according to claim 1, wherein the control station is local to the worksite.

7. The system according to claim 6, further comprising a compaction machine, wherein the compaction machine is part of the control station.

8. The system according to claim 1, wherein the control station is cloud-based and remote from the worksite.

9. The system according to claim 1,
wherein the control station wirelessly receives the moisture data from a machine at the worksite, and
wherein the machine at the worksite receives the moisture data outputted from each of the wireless moisture sensors only when the machine is within a predetermined distance away from the wireless moisture sensor.

10. The system according to claim 9, wherein the machine is a compaction machine or an unmanned aerial vehicle (UAV).

11. The system according to claim 1, wherein the circuitry of the control station is configured to estimate a future moisture content of the soil associated with one or more of the wireless moisture sensors based on weather at the worksite and soil characteristics around the one or more wireless moisture sensors, under a condition that the moisture data from the one or more wireless moisture sensors has not been received by the control station for a predetermined amount of time.

12. A method comprising:
wirelessly receiving, at processing circuitry, moisture data outputted from one or more wireless moisture sensors at least partially embedded in a material to be compacted by a compaction machine at a jobsite;
identifying, using the processing circuitry, whether the material around each of the one or more wireless moisture sensors qualifies for compaction according to predetermined criteria, the predetermined criteria for compaction by the compaction machine including whether a moisture content of the material around each of the one or more wireless moisture sensors is within a predetermined moisture threshold range; and
responsive to the material around at least one of the one or more wireless moisture sensors failing to qualify for compaction, wirelessly outputting, using the processing circuitry, signaling to direct a water truck to provide water to a surface of the material associated with a location of the at least one wireless moisture sensor.

13. The method according to claim 12, further comprising wirelessly outputting, using the processing circuitry, information to the compaction machine to compact the surface of the material associated with a location of the at least one wireless moisture sensor.

14. The method according to claim 12,
wherein each of the one or more wireless moisture sensor is one of a tag or a stake, the tag being fully embedded in the material and the stake being partially embedded in the material, and
wherein the processing circuitry wirelessly receives the moisture data indirectly from the one or more wireless moisture sensors, via a machine at the jobsite different from the water truck.

15. The method according to claim 14, wherein the machine directly receives the moisture data outputted from the one or more wireless moisture sensors, only when the machine is within a predetermined distance away from each of the one or more wireless moisture sensors.

16. The method according to claim 12, further comprising estimating, using the processing circuitry, future moisture content of the material associated with at least one of the one or more wireless moisture sensors based on weather and/or material characteristics, under a condition that the moisture data from the at least one wireless moisture sensor has not been received by the processing circuitry for a predetermined amount of time.

17. A non-transitory computer-readable storage medium having stored thereon instructions that, when executed by one or more processors, cause the one or more processors to perform a method comprising:
wirelessly receiving moisture data outputted from at least one wireless moisture sensor at least partially buried in a material to be compacted by a compactor at a worksite;
determining, based on the moisture data, whether the material at each said at least one wireless moisture sensor is or will soon be below a predetermined moisture threshold range; and
responsive to said determining indicating that the material at each said at least one wireless moisture sensor is or will soon be below the predetermined moisture threshold range, outputting signaling to direct a water truck to provide water to a surface of the material associated with each said at least one wireless moisture sensor.

18. The non-transitory computer-readable storage medium according to claim 17, wherein the method further comprises, after the water truck has provided water to the surface of the material associated with each said at least one wireless moisture sensor, wirelessly outputting information to the compactor to compact the surface of the material associated with each said at least one wireless moisture sensor according to a timing whereby the material will be within the predetermined moisture threshold range when the compactor compacts the surface of the material.

19. The non-transitory computer-readable storage medium according to claim 17,
wherein each said at least one wireless moisture sensor is one of a tag or a stake, the tag being fully embedded in the material and the stake being partially embedded in the material,
wherein the moisture data is received indirectly from each said at least one wireless moisture sensor, from a machine at the worksite different from the water truck, and
wherein the machine receives the moisture data outputted from the at least one wireless moisture sensor only when the machine is within a predetermined distance away from the at least one wireless moisture sensor.

20. The non-transitory computer-readable storage medium according to claim 17, wherein the method further comprises estimating future moisture content of the material associated with each said at least one wireless moisture sensor based on weather and material characteristics.
